(19) United States
(12) Patent Application Publication
(10) Pub. No.: US 2006/0204561 A1
(43) Pub. Date: Sep. 14, 2006

(54) Device for delivery of TRPV1 agonists

(76) Inventors: Naweed Muhammad, Fremont, CA (US); Gene C. Jamieson, Boulder Creek, CA (US); Keith R. Bley, Mountain View, CA (US)

Correspondence Address: MORRISON & FOERSTER LLP, 755 PAGE MILL RD, PALO ALTO, CA, 94304-1018, US

(21) Appl. No.: 11/354,349

(22) Filed: Feb. 14, 2006

Related U.S. Application Data
(60) Provisional application No. 60/652,923, filed on Feb. 14, 2005.

(51) Int. Cl. A61K 36/81; A61K 31/16; A61F 13/02

(52) U.S. Cl. 424/448; 424/760; 514/630

(57) ABSTRACT

Described here are drug delivery devices including an occlusive backing layer and a drug depot containing a TRPV1 agonist and a non-hydrophilic solvent. The drug depot may take various forms, such as an adhesive polymeric matrix, liquid reservoir, or microreservoir droplets. Methods of making and using the drug delivery devices are also described.

### CROSS-REFERENCE TO RELATED APPLICATIONS

This application claims priority to U.S. Provisional Patent Application Ser. No. 60/652,923, filed Feb. 14, 2005, which is hereby incorporated by reference in its entirety.

### FIELD

The devices and methods described here are in the field of drug delivery. More specifically, the described devices and methods relate to dermal delivery of capsaicin and other TRPV1 agonists for alleviating pain.

### BACKGROUND

The transient receptor potential vanilloid 1 receptor (TRPV1) is a capsaicin-responsive ligand-gated cation channel selectively expressed on small, unmyelinated peripheral nerve fibers (cutaneous nociceptors) (see, Caterina and Julius, 2001, “The Vanilloid Receptor: A Molecular Gateway to the Pain Pathway,” Annu Rev Neurosci, 24:487-517; and Montell et al., 2002, “A unified nomenclature for the superfamily of TRP cation channels,” Mol Cell, 9:229-31). When TRPV1 is activated by agonists such as capsaicin and other factors such as heat and hydrogen ions, calcium enters the cell and pain signals are initiated.

Capsaicin and other TRPV1 agonists may be effective for amelioration of a plurality of conditions. For example, capsaicin may be used to treat various types of pain, such as neuropathic and chronic pain (including pain associated with diabetic neuropathy, postherpetic neuralgia, HIV infection, traumatic injury, complex regional pain syndrome, trigeminal neuralgia, erythromelalgia and phantom pain), pain produced by mixed nociceptive and/or neuropathic mixed etiologies (e.g., cancer, osteoarthritis, fibromyalgia, low back pain, inflammatory hyperalgesia, vulvar vestibulitis or vulvodynia, sinus polyps interstitial cystitis, neurogenic or overactive bladder, prostatic hyperplasia, rhinitis, surgery, trauma, rectal hypersensitivity, burning mouth syndrome, oral mucositis, herpes (or other viral infections), prostatic hypertrophy and headaches) (see, Szallasi and Blumberg, 1999, “Vanilloid (Capsaicin) Receptors and Mechanisms,” Pharm Revs, 51:159-211; Backonja et al., “A Single One Hour Application of High-Concentration Capsaicin Patches Leads to Four Weeks of Pain Relief in Postherpetic Neuralgia Patients” American Academy of Neurology, 2003 (meeting abstract); Berger et al., 1995, J Pain Symptom Management 10:243-8). Additionally, capsaicin may be used to treat skin conditions such as dermatitis, pruritis, itch, psoriasis, warts and wrinkles, as well as conditions such as tinnitus and cancers (especially skin cancers) (see, Bernstein et al., 1986, “Effects of Topically Applied Capsaicin on Moderate and Severe Psoriasis Vulgaris,” J Am Acad Dermatol 15:504-507; Ellis et al., 1993, “A Double-Blind Evaluation of Topical Capsaicin in Pruritic Psoriasis,” J Am Acad Dermatol 29:438-42; Saper et al., 2002, Arch Neurol 59:990-4; and Vass et al., 2001, Neuroscience 103:189-201; Moller, 2000, “Similarities between severe tinnitus and chronic pain” J Am Acad Audiol. 11:115-24).

Numerous drug delivery devices have sought to deliver capsaicin. For example, U.S. Pat. No. 6,239,180 to Robbins describes the use of a drug delivery device comprising capsaicin and/or a capsaicin analog at a concentration of greater than 5% by weight for treatment of neuropathic pain. WO 2004/089361 to Muller describes a topical patch comprising a therapeutic compound-impermeable backing layer, a polysiloxane matrix containing capsaicin and an amphiphilic solvent, and a protective film to be removed before use. Additionally, U.S. Publication No. 2005/0090557 to Muhammad et al. describes the delivery and pharmacological properties of topical liquid formulations of TRPV1 agonists. However, none of these references describe the delivery of capsaicin with the aid of non-hydrophilic penetration enhancers in patch formulations. Specifically, none of these references describe the use of an occlusive backing to enhance delivery of water-insoluble compounds through the skin.

The use of an occlusive backing layer to stop/minimize escape of water from the skin, or in other words, to substantially prevent transepidermal water loss (TEWL), is known to those skilled in the art. It is also known that retention of this water results in hydration of stratum corneum and in turn increases skin permeability to penetrants such as drug molecules (see, Roberts et al. (1993) Water: The Most Natural Penetartion Enhancer. In: Pharmaceutical Skin Penetration Enhancement, Eds. K. A. Walter and J. Hadgraft. Marcel Dekker, New York, pp. 1-30). However, use of this escaping water and non-hydrophilic penetrations enhancers to increase the thermodynamic activity of the drug depot has not been described.

Accordingly, it would be desirable to have occlusive patches that include non-hydrophilic penetration enhancers for delivery of capsaicin and other TRPV1 agonists for the treatment of pain and other conditions.

### BRIEF SUMMARY

Described here are drug delivery devices and methods for administering capsaicin and other TRPV1 agonists. In general, the drug delivery devices include a therapeutically effective amount of an active agent for dermal delivery that is useful for treating pain. The devices are usually configured for topical application and provide local administration of drug to the area in need of treatment.

The drug delivery devices may be formulated as any conventional patch type, e.g., polymeric matrix, adhesive, or reservoir, and made by methods well known in the art. In all instances, however, the devices include an occlusive backing that substantially prevents transepidermal water loss and a non hydrophilic penetration enhancer.

The patches typically include capsaicin, but may also be formulated to incorporate other TRPV1 agonists such as, but not limited to, capsaicinoids, capsaicin analogs, and capsaicin derivatives. The patches may include a TRPV1 agonist in an amount of at least about 0.04%, at least about 2%, at least about 4%, at least about 6%, at least about 8%, at least about 10%, at least about 20%, or at least about 30% by weight of the drug depot of the device. The particular non-hydrophilic penetration enhancer employed in the patches will also vary, depending on such factors as device type (e.g., polymeric matrix, liquid reservoir, etc.), adhesive used, and the like, but in all instances will have a ClogP value greater than 1.0.

The drug delivery devices may be used to treat various conditions. For example, they may be used to treat various types of pain such as, but not limited to, neuropathic and chronic pain (including pain associated with diabetic neuropathy, postherpetic neuralgia, HIV infection, traumatic injury, complex regional pain syndrome, trigeminal neuralgia, erythromelalgia and phantom pain), pain produced by mixed nociceptive and/or neuropathic mixed etiologies (e.g., cancer, osteoarthritis, fibromyalgia, low back pain, inflammatory hyperalgesia, vulvar vestibulitis or vulvodynia, sinus polyps interstitial cystitis, neurogenic or overactive bladder, prostatic hyperplasia, rhinitis, surgery, trauma, rectal hypersensitivity, burning mouth syndrome, oral mucositis, herpes (or other viral infections), prostatic hypertrophy, and headaches). The drug delivery devices may also deliver an active agent to treat conditions such as dermatitis, pruritis, itch, psoriasis, warts and wrinkles, as well as conditions such as tinnitus and cancers (especially skin cancers).

Methods for treating pain are also described. In some variations, the methods include applying a drug delivery device having a TRPV1 agonist, a non-hydrophilic penetration enhancer with a ClogP value greater than 1.0, and an occlusive backing to the skin or mucous membrane of a subject, and delivering a therapeutically effective amount of the TRPV1 agonist to alleviate the pain. The TRPV1 agonist may be delivered over time periods of at least about 15 minutes, or time periods of greater than about 15 minutes, greater than about 30 minutes, greater than about 1 hour, greater than about 4 hours, greater than about 6 hours, greater than about 12 hours, greater than about 18 hours, or greater than about 24 hours or more.

### BRIEF DESCRIPTION OF THE DRAWINGS

FIG. 1 shows a microreservoir type of drug delivery device including an impermeable backing layer 1, a self-adhesive matrix containing an active agent dispersed in the form of microreservoir droplets 2, and a protective film 3 to be removed before use.

FIG. 2 depicts a monolithic type of drug delivery device including an impermeable backing layer 1, a monolithic matrix acting as an active agent depot whereby active agent has been dissolved and/or dispersed in a polymer matrix forming a gel-like or solid mass 2, an adhesive layer 4, and a protective film to be removed before use 3. It may have an optional diffusion-rate-controlling membrane (not shown) between 2 and 4.

FIG. 3 illustrates a monolithic type of drug delivery device comprising an impermeable backing layer 1, a monolithic matrix acting as an active agent depot whereby active agent has been dissolved and/or dispersed in a polymer matrix forming a gel-like or solid mass 2, a diffusion-rate-controlling membrane 5, an adhesive layer 4 at the periphery such that the diffusion-rate-controlling membrane comes in direct contact with the skin surface on one side and monolithic matrix on the other side, and a protective film 3 to be removed before use. It should be noted that impermeable backing layer 1 is heat-sealed with diffusion-rate-controlling membrane 5 thus creating a pocket in which monolithic matrix is enclosed.

FIG. 4 shows a liquid reservoir type of drug delivery device comprising an impermeable backing layer 1, a liquid reservoir acting as an active agent depot whereby active agent has been dissolved, completely or partially, in a penetration enhancer or a mixture thereof 2, a diffusion-rate-controlling membrane 5, an adhesive layer 4, and a protective film 3 to be removed before use.

FIG. 5 depicts a liquid reservoir type of drug delivery device comprising an impermeable backing layer 1, a liquid reservoir acting as an active agent depot whereby active agent has been dissolved, completely or partially, in a penetration enhancer or a mixture thereof 2, an diffusion-rate-controlling membrane 5, an adhesive layer 4 at the periphery such that the diffusion-rate-controlling membrane comes in direct contact with the skin surface on one side and liquid reservoir on the other side, and a protective film 3 to be removed before use 3. It should be noted, again, that impermeable backing layer 1 is heat-sealed with diffusion-rate-controlling membrane 5 thus creating a pocket in which the active agent containing liquid reservoir 2 is enclosed.

FIG. 6 shows the in vitro release into deionized water of capsaicin from six microreservoir patches over 18 hours. Each patch contained a different capsaicin concentration. The following capsaicin concentrations (by weight of the drug depot) were tested: 0.04%, 2%, 4%, 6%, 8%, and 10%.

FIG. 7 shows the in vitro release into deoionized water of capsaicin from six monolithic patches over 24 hours. Each patch contained a different capsaicin concentration. The following capsaicin concentrations (by weight of the drug depot) were tested: 0.04%, 2%, 4%, 6%, 8%, and 10%.

FIG. 8 shows a selective portion of the graph in FIG. 7 to better illustrate shape of the curves at early time-points (i.e., 30 minutes, 1 hour and 3 hours).

### DETAILED DESCRIPTION

The drug delivery devices described here may be of any configuration so long as they include a non-hydrophilic penetration enhancer and deliver a therapeutically effective amount of an active agent for an indicated condition, e.g., pain or a skin condition. In general, the devices are patches that are configured to have an occlusive backing layer, a non-hydrophilic penetration enhancer, an active agent partially or completely dissolved in the non-hydrophilic penetration enhancer such that the resulting composition forms drug dispersed in an adhesive, or is a liquid reservoir, or a monolith matrix, etc., and a peelable release liner.

As previously mentioned, incorporation of a non-hydrophilic penetration enhancer into an occlusive patch is believed to enhance the thermodynamic activity of the drug depot. Another advantage of using a non-hydrophilic penetration enhancer relates to the decreased effect its inclusion has on hydrolysis of the active agents. Esters and amides are particularly sensitive to hydrolysis. Capsaicin and capsaicinoids are amides. It is, therefore, desirable to have anhydrous formulations of capsaicin-containing drug products in order to ensure longer shelf lives. Also, the hygroscopicity exhibited by amphiphilic and hydrophilic solvents makes it difficult to assure that the drug products' ingredients will be water-free during procurement, storage, and manufacturing. For instance, the drying of patches to evaporate solvents used to dilute the adhesives is often conducted at relatively low temperatures (i.e., up to 40° C.) which can not effectively drive off any water vapors present in the formulations. This consideration renders hydrophilic and amphiphilic skin penetration enhancers less desirable for use in many different types of dosage forms, including dermal and transdermal patches.

In addition, amphiphilic and hydrophilic skin penetration enhancers such as ethanol, acetone, and DMSO are known to partition preferentially into intracellular domains of the stratum corneum. In contrast, non-hydrophilic skin penetration enhancers are more likely to intercalate into the structured lipids of the stratum corneum and disrupt the packing of the horny cells without actually permeabilizing the horny cells (see, Rolf Daniels, “Strategies for skin penetration enhancement,” Skin Care Forum, Issue 37, August 2004). Accordingly, lower levels of skin damage or irritation may be associated with the use of non-hydrophilic skin penetration enhancers.

As used herein, the terms “active agent,” “active,” “drug,” or “therapeutic compound” are used interchangeably, and refer to capsaicin, other TRPV1 agonists, or combinations thereof. By “therapeutically effective amount” it is meant an amount of drug effective to treat pain or any other indicated condition. Furthermore, as used herein, the term “drug depot” refers to that portion or layer of the drug delivery device in which the drug is incorporated, and excludes the occlusive backing layer, release liner, and diffusion-rate-controlling membrane. It also excludes adhesive when the drug is not present in the adhesive mass.

The terms “penetration enhancer” and “solvent” are used interchangeably, and refer to any compound (liquid or solid) which enhances penetration of a molecule (e.g., a drug molecule) into the skin, excluding the following: butanediols, such as 1,3-butanediol, dipropylene glycol, tetrahydrofurfuryl alcohol, diethylene glycol dimethyl ether, diethylene glycol monoethyl ether, diethylene glycol monobutyl ether, propylene glycol, dipropylene glycol, carboxylic acid esters of tri- and diethylene glycol, polyethoxylated fatty alcohols of 6-18 carbon atoms, 2,2-dimethyl-4-hydroxymethyl-1,3-dioxolane (Solketal®), and mixtures thereof.

Furthermore, as used herein, the term “treat”, “treating”, or “treatment” refers to the resolution or reduction of pain or symptoms or the underlying cause of a condition, or prevention of a condition.

Conditions for which capsaicin or other TRPV1 agonist treatment may be indicated include, but are not limited to, neuropathic pain (including pain associated with diabetic neuropathy, postherpetic neuralgia, HIV/AIDS, traumatic injury, complex regional pain syndrome, trigeminal neuralgia, erythromelalgia and phantom pain), pain produced by mixed nociceptive and/or neuropathic mixed etiologies (e.g., cancer, osteoarthritis, fibromyalgia and low back pain), headache, inflammatory hyperalgesia, interstitial cystitis, and skin conditions such as dermatitis, pruritis, itch, psoriasis and warts. Generally, the capsaicin- or other TRPV1 agonist-containing drug delivery devices can be used to treat any condition for which topical administration of capsaicin is beneficial. As used herein, the term “topical,” “topical administration” and “topically” refer to local administration of capsaicin or other TRPV1 agonists to the skin or mucous membrane.

At the time of application, the release liner is first removed from the patch. The patch is then placed on the skin or mucosal surface to be treated, with the occlusive backing being opposite the skin or mucosal surface. If desired, gentle pressure may be applied to the patch to assure patch adherence. The release liner is usually made from a drug-impermeable material, and is configured to be a disposable element which serves only to protect the device prior to application.

I. Drug Delivery Devices

As mentioned above, the drug delivery devices described herein may be of any form, so long as they include an occlusive backing, a non-hydrophilic penetration enhancer, and deliver a therapeutically effective amount of a drug.

In general, the backing may be adapted to provide varying degrees of flexibility to the device, according to the needs of the desired application. The functions of the backing layer are to provide an occlusive barrier that prevents loss of transepidermal water, the drug and the non-hydrophilic penetration enhancer(s) to the environment, and to protect the patch. The material chosen for the backing should exhibit minimal drug compound and enhancer permeability and should not be incompatible with them or with the adhesive. Ideally, the backing material should be capable of forming a support onto which the drug-containing mixture can be cast and to which it will bond securely during manufacturing, storage, and use. Examples of such materials include, but are not limited to, polyurethane, polyethylene, ethylene vinyl acetate, pigmented polyethylene plus polyester with/without aluminum vapor coating, and polyester with ethylene vinyl acetate copolymer. Examples of commercial brands are CoTran™ and Scotchpak™ backing films. As an alternative to casting the matrix directly on the backing layer, the matrix may be cast separately and later stuck to the backing material.

In one variation, the drug delivery device is a matrix system. Matrix systems are characterized (in the simplest case) by an occlusive backing layer impermeable to the active agent (i.e., compound to be delivered to the subject), an active agent-containing layer, and a release liner to be removed before use. The active agent-containing layer contains the active agent in completely or partially dissolved form and is ideally self-adhesive. The matrix systems may be composed of a number of layers and can include a control membrane. Adhesive polymers suitable for use in this type of system include, but are not limited to, polyacrylates, polysiloxanes, polyurethanes, polyisobutylenes, and combinations thereof. Matrix systems may be multiple layers in which concentrations of active agent differ in different layers; such construction serves as a means to modify the release profile of active agent over time.

The adhesive used in an adhesive matrix type delivery device may be selected from a variety of adhesives available commercially and known to those skilled in the art. For example, common adhesives are those based on polyisobutylene, polyacrylate, and ploysiloxane. The adhesives can even be hydrophilic such as high molecular weight polyethylene oxide or polyvinylpyrrolidone. The selection of the adhesive is critical to realize a functioning adhesive matrix type drug delivery device. The non-hydrophilic penetration enhancers and the drug are loaded directly into the adhesive and so the adhesive must retain its chemical, viscoelastic, and adhesive properties in the presence of these additives. The adhesive properties include sufficient tack for good instantaneous adhesion to the skin as well as maintenance of adhesion. It is often seen that adhesives become stringy and gooey in the presence of skin permeation enhancers, leading to cohesive failure and residual adhesive left on the patient's skin after removal of the device. In some cases, the device looses adhesion altogether and falls off. The loss of tack and adhesion properties generally dictates and limits the amount and type of non-hydrophilic enhancers that can be loaded into the adhesive matrix type delivery device. Some acrylate based adhesives, such as those available from Avery and National Starch and Chemical Company, are able to withstand relatively high loadings of non-hydrophilic enhancers, both solvent-type and plasticizing type. In addition, Bio-PSAs from Dow-Corning are also compatible with non-hydrophilic penetration enhancers.

FIG. 1 shows an adhesive matrix type patch that includes an occlusive backing layer 1 and an adhesive matrix layer 2, which serves both as a depot for the active agent and a means of adhering the device to the skin. The active agent-containing adhesive matrix layer 2 may include the drug dispersed in the adhesive polymer matrix 2. As used herein, the term “dispersed” refers to the distribution of the drug throughout the matrix. The drug may be dispersed in a dissolved and/or undissolved state.

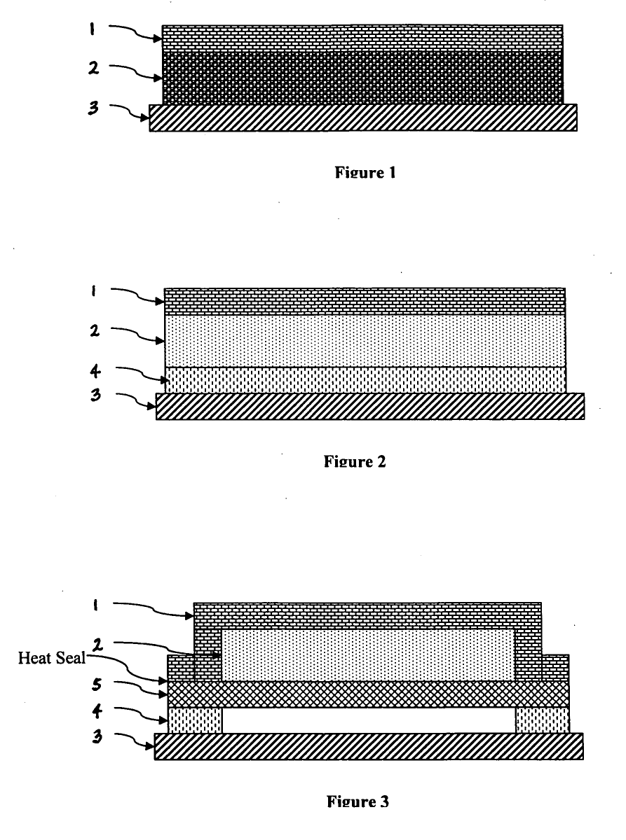

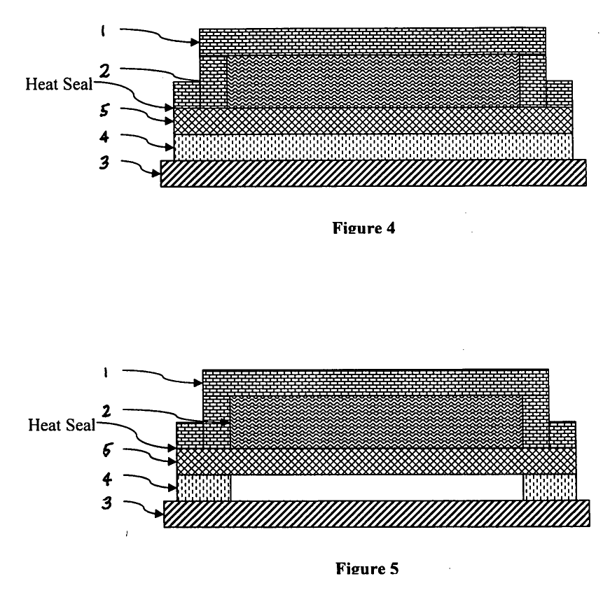

In another variation, the drug delivery device is a monolithic matrix device, as shown in FIGS. 2 and 3. In a monolithic device, material other than the adhesive serves as the drug depot. For these delivery devices, hydrogel materials may be used as the matrix material. For example, polyurethane, gelatin, and pectin may be used. The drug depot may also be formed in materials like ethyl cellulose, hydroxypropyl cellulose (with consistencies ranging from a gel-like to a solid mass). Such drug depots can contain relatively large volumes of non-hydrophilic penetration enhancers or mixtures thereof, necessary for effective drug delivery. In the case of a drug depot having gel-like consistency, a diffusion-rate-controlling membrane may be included to interface with the skin surface and depot. In the case of a firm/solid depot, the use of diffusion-rate-controlling membrane is also optional.

Referring now to FIGS. 2 and 3, the monolithic matrix type drug delivery device comprises an impermeable backing layer 1, a monolithic matrix layer 2, an optional diffusion-rate-controlling membrane 5, and an adhesive layer 4. The backing 1, membrane 5, and adhesive layer 4 are selected as described above. One of the functions of the diffusion-rate-controlling membrane is to provide structural support for the adhesive layer which simplifies the manufacturing of the device. The monolithic matrix layer is distinguished from the adhesive matrix of FIG. 1 where the monolith serves as the drug reservoir and the skin adhesive interfaces between the release liner and the monolith.

In some instances, as shown in FIG. 3, the adhesive layer 5, may be applied to the periphery of the patch so as not to come in contact with non-hydrophilic penetration enhancers. This is particularly desirable in situations where a high loading and/or the nature of non-hydrophilic penetration enhancers may interfere with adhesion.

Monolith matrix materials are generally those materials capable of holding a large volume of liquid such as the non-hydrophilic penetration enhancers employed. Suitable materials are polymers such as hydroxy ethyl methacrylate (HEMA) ethyl methacrylate (EMA) blends, polyvinyl alcohols, polyvinyl pyrrolidine, gelatin, pectin, and other hydrophilic materials. Microporous particles may be incorporated into the polymer monolith to hold the solvent type enhancers used. The use of microporous particles in transdermal patches is disclosed by Katz et al. in U.S. Pat. No. 5,028,535, Sparks et al. in U.S. Pat. No. 4,952,402, and Nuwayser et al. in U.S. Pat. No. 4,927,687, all of which are hereby incorporated by reference in their entirety.

The drug and non-hydrophilic penetration enhancers may be loaded into the microporous particles before incorporation into the hydrophilic polymer. The particles may then be evenly dispersed throughout the matrix by mixing. At high loadings of particles, the release of therapeutic compound and non-hydrophilic penetration enhancer is enhanced due to the formation of channels in the polymer matrix. Suitable microporous particles are diatomaceous earth, silica, cellulose acetate fibers from Hoechst Celanese, and Polytrap® from Dow Corning.

The monolith layer may be prepared as follows. First a solution of the adhesive polymer is obtained or prepared. Another solution or dispersion of the drug in non-hydrophilic penetration enhancers is prepared and mixed until the drug is dissolved or evenly dispersed. The viscosity of the drug/non-hydrophilic penetration enhancer solution or dispersion may then be adjusted by adding and mixing viscosity enhancing agents. For example, ethyl cellulose and hydroxypropyl cellulose may be employed to adjust viscosity. The resulting solution or dispersion is then added to the adhesive polymer solution and the mixture is homogenized such that the drug solution/dispersion is distributed in the adhesive in the form of droplets. A suitable solvent, which is later removed by drying, can be added to this mixture to facilitate homogenization and/or casting. Examples of such solvents are n-heptane and ethyl acetate. The homogenized adhesive mass or solution may then be poured into a mold or cast alone or on the desired backing material. The casting is then left for the solvent to evaporate at room temperature or in an oven at a slightly elevated temperature. A vacuum or air current can be employed to facilitate solvent evaporation. After solvent evaporation, the adhesive matrix takes the form of an adhesive polymer film typically having a thickness in the range of about 30 to 200 μm.

In yet another variation, the drug delivery device is a reservoir system. In a reservoir system, a pouch (formed by heat-sealing of an impermeable backing layer with a diffusion-rate-controlling membrane) contains the drug, dissolved completely or partially, in a liquid. Exemplary liquid reservoir systems are shown in FIGS. 4 and 5. As used herein the term “diffusion-rate-controlling membrane” generally refers to a semi-permeable membrane that limits the rate of release of a drug from the delivery device. The membrane can be a microporous film or a nonporous partition membrane. The side facing the skin is also protected in this drug delivery device design by a film that has to be removed before use.

Referring now to FIGS. 4 and 5, the reservoir type drug delivery device includes, from the non-skin-facing side to skin-facing side of device, an impermeable backing layer 1, a drug reservoir (drug depot) 2, a diffusion-rate-controlling membrane 5, and an adhesive layer 4. The backing layer 1, may be the same as that described for the adhesive matrix type delivery device above. The reservoir may take various forms, for example, the drug may be dissolved in a non-hydrophilic penetration enhancer or mixture thereof, gelled or ungelled. Alternatively, the drug/non-hydrophilic enhancer(s) mixture may be conveniently contained in the pores of a pad or foam material such as polyurethane foam. One function of the reservoir is to keep the drug and non-hydrophilic enhancer(s) in good contact with the membrane layer.

The diffusion-rate-controlling membrane 5 in its most simple function provides mechanical support for the adhesive layer 4. The membrane layer and backing layer are heat-sealed at their peripheral edges to form a pouch which encloses the drug reservoir. As used herein, the term “peripheral edges” of the membrane and backing layers refers to the areas that are sealed together to define the drug reservoir boundaries. Therefore, extraneous membrane and backing layer material may extend outwardly from the drug reservoir and peripheral edges. The membrane and adhesive layers must be freely permeable to therapeutic compound and to the enhancers. As such, the membrane layer should offer diffusional resistance as tailored by the choice of membrane. Generally, diffusion-rate-controlling membranes have a known MVTR (moisture vapor transmission rate) value described as g/cm²/24 hr. Without being limiting, an exemplary MVTR value of 15 to 100 g/cm²/24 hr is generally suitable. MTVR values outside this range may be warranted depending, for example, on the physicochemical properties the drug, its concentration in the reservoir, thermodynamic properties of the reservoir and drug dose, and desired rate of administration.

An advantage of a reservoir system is that the saturation solubility of the drug can be adjusted more readily by modifying the non-hydrophilic penetration enhancer(s) included in the reservoir. For thermodynamic reasons, it is advantageous for the release of the drug in and on the skin if it is present in the drug-containing parts of the drug delivery device at a concentration that is not too far below the saturation concentration. The uptake capacity of the drug delivery device for the amount of drug needed can be adjusted in a wide range to suit particular needs by adjusting the amount of drug solution and extent of saturation of the solution. For example, saturation of the drug solution may range from nearly-saturated to supersaturated, or the solution may contain an undissolved fraction of the drug. Nearly-saturated or supersaturated drug solutions are high thermodynamic activity systems that enhance the tendency of a drug to be released.

In a further variation, the drug delivery device is a microreservoir system. Microreservoir systems are generally viewed as a combination of the matrix and reservoir type of systems. In a microreservoir system, a liquid ranging from a very low to very high viscosity contains a drug(s) in a completely or partially dissolved state and is dispersed as a fine droplets into a solid adhesive matrix. If desired, viscosity of the liquid component of the system may be enhanced by using viscosity enhancing agents such as ethyl cellulose, hydroxypropyl cellulose or a high molecular weight polyacrylic acid or its salt and/or derivatives such as esters.

In one variation, a microreservoir drug delivery device includes an occlusive backing layer, a self-adhesive matrix comprising microreservoirs of solution of drug, partially or completely dissolved in a non-hydrophilic penetration enhancer, and a protective film (release liner) to be removed before use of the device. The drug (e.g., capsaicin) in the microreservoir system is dissolved completely or partially and the resulting solution and/or mixture is gelled with a viscosity enhancing agent, for example, ethyl cellulose and/or hydroxypropyl cellulose, such that when it is mixed with an adhesive or mixture of adhesives, it forms discrete globules which are distributed throughout the adhesive mass forming a “microreservoir” of drug. For the purposes of the devices and methods described herein, the terms “microreservoir” and “microreservoir droplets” refer to microdispersed droplets that include a drug, and a non-hydrophilic penetration enhancer or mixture of non-hydrophilic penetration enhancers, and may optionally include a viscosity enhancer. The term “microreservoir system” is a collection of these microreservoir droplets dispersed in an adhesive mass (e.g., a pressure sensitive adhesive (PSA)), with or without additional components.

As used herein, the terms “adhesive” and “adhesive mass” refer to materials capable of adhering to the skin as well as to occlusive or mpermeable backing films or diffusion-rate-controlling membranes. The term “pressure sensitive adhesive” refers to an adhesive (e.g., polysiloxane, polyacrylate, or polyisobutylene) which adheres to the skin surface when pressed onto it. Generlly, the polysiloxane- or polyacrylate or polyisobutylene-based self-adhesive matrix will be configured to include the active agent in an amount of at least about 0.001% by weight of adhesive mass, at least about 0.01% by weight of adhesive mass, at least about 0.1% by weight of adhesive mass, at least about 1% by weight of adhesive mass, at least about 3% by weight of adhesive mass, at least about 5% by weight of adhesive mass, at least about 10% by weight of adhesive mass, at least about 15% by weight of adhesive mass, at least about 20% by weight of adhesive mass, or at least about 30% by weight of adhesive mass.

Surprisingly, we have now found that a drug delivery device for treating chronic pain or skin conditions containing a high concentration of capsaicin or other TRPV1 agonist can be improved by including a non-hydrophilic penetration enhancer in the device which has a ClogP value of 1.0 or higher. The term “ClogP” refers to a water/octanol partition coefficient as calculated by “ClogP for Windows” software, version 4.0, by Biobyte Corp. (Claremont, Calif., USA). Apart form the intrinsic ability of such penetration enhancers to enhance dermal and transepidermal delivery of drugs, transepidermal water loss (TEWL) also plays a role in the function of the drug delivery devices described in this invention. TEWL refers to loss of water from the skin surface and is a distinctly different mechanism than water loss by sweat glands. It is a continuous process and is considered to be a parameter to evaluate integrity of the skin (i.e., damaged or permeabilized skin exhibits higher TEWL). When penetration enhancer-containing drug reservoirs (i.e., patches) trap and retain water leaving the skin surface due to TEWL, the thermodynamic activity of the drug substance can be enhanced if the drug substance has low solubility in water. This can, in consequence, result in enhanced release of therapeutic compound(s) from the delivery device.

Those skilled in the art appreciate that use of amphiphilic or hydrophilic skin penetration enhancers is very common in such delivery devices. However, in such known devices, water lost from the skin surface is trapped and becomes part of the drug reservoir, owing to the miscibility of water with the amphiphilic or hydrophilic skin penetration enhancers contained therein. In consequence, such systems fail to take advantage of water resulting from the prevention of TEWL. Also, such hygroscopic systems are amenable to picking up atmospheric water vapor during manufacturing, leading to hydrolytic degradation of the drug during manufacture and/or shelf life storage.

In contrast, the drug delivery devices contemplated herein utilize non-hydrophilic skin penetration enhancers in which the drug(s) has been solubilized (completely or partially) and thus form a drug reservoir. In such drug delivery devices, when water lost from the skin surface is trapped by the reservoir, the skin penetration enhancers have increased thermodynamic activity due to their immiscibility with water. The result is that the release of the skin penetration enhancers from the reservoir is enhanced, and thus more therapeutic compound is delivered into and perhaps through the skin.

A. TRPV1 Agonists

TRPV1 agonists useful in the present invention include, but are not limited to, capsaicin, capsaicin analogs and derivatives, and other low molecular weight compounds (i.e., MW<1000) that are agonistic to the TRPV1. Capsaicin can be considered the prototypical TRPV1 agonist. Capsaicin (also called 8-methyl-N-vanillyl-trans-6-nonenamide; (6E)-N-[(4-hydroxy-3-methoxyphenyl)methyl]-8-methylnon-6-enamide; N-[(4-hydroxy-3-methoxyphenyl)methyl]-8-methyl-(6E)-6-nonenamide; N-(3-methoxy-4-hydroxybenzyl)-8-methylnon tran-6-enamide; (E)-N-[(4-hydroxy-3-methoxyphenyl)methyl]-8-methyl-6-nonenamide) has the following chemical structure:

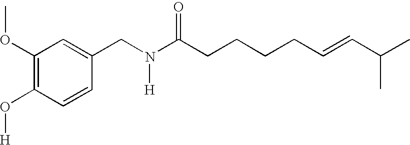

Suitable capsaicin analogs for use in the drug delivery devices include naturally occurring and synthetic capsaicin derivatives and analogs (“capsaicinoids”) such as, for example, those described in U.S. Pat. No. 5,762,963, which is incorporated herein by reference in its entirety.

In addition to capsaicin, a variety of capsaicin analogs and derivatives, and other TRPV1 agonists may be administered. Vanilloids, such as capsaicinoids, are examples of useful TRPV1 agonists. Exemplary vanilloids suitable for use with the devices and methods described herein include N-vanillyl-alkanedienamides, N-vanillyl-alkanedienyls, N-vanillyl-cis-monounsaturated alkenamides, capsaicin, dihydrocapsaicin, norhydrocapsaicin, nordihydrocapsaicin, homocapsaicin, and homodihydrocapsaicin.

The TRPV1 agonist may also be a compound lacking the vanillyl function, such as piperine or a dialdehyde sesquiterpene (for example warburganal, polygodial, or isovelleral). In one embodiment, the TRPV1 agonist is a triprenyl phenol, such as scutigeral. Additional exemplary TRPV1 agonists are described in U.S. Pat. Nos. 4,599,342; 5,962,532; 5,762,963; 5,221,692; 4,313,958; 4,532,139; 4,544,668; 4,564,633; 4,544,669; 4,493,848; 4,532,139; 4,564,633; and 4,544,668; and PCT publication WO 00/50387, each of which are incorporated by reference in their entirety. Other useful TRPV1 agonists include pharmacologically active gingerols, piperines, shogaols, and more specifically guaiacol, eugenol, zingerone, civamide, nonivamide, nuvanil, olvanil, NE-19550, NE-21610, and NE-28345 (see Dray et al., 1990, Eur. J. Pharmacol 181:289-93 and Brand et al., 1990, Agents Actions 31:329-40), resiniferatoxin, resiniferatoxin analogs, and resiniferatoxin derivatives (e.g., tinyatoxin). In addition, any active geometric- or stereo-isomer of the forgoing agonists may be used with the devices and methods described herein.

Other suitable TRPV1 agonists are vanilloids that have TRVP1 receptor-binding moieties such as mono-phenolic mono-substituted benzylamine amidated with an aliphatic cyclized, normal or branched substitution. Still other suitable TRPV1 agonists for use with the devices and methods described herein can be readily identified using standard methodology, such as that described in U.S. patent publication US20030104085, which publication is hereby incorporated by reference in its entirety. Useful assays for identification of TRPV1 agonists include, without limitation, receptor binding assays; functional assessments of stimulation of calcium influx or membrane potential in cells expressing the TRPV1 receptor, assays for the ability to induce cell death in such cells (e.g., selective ablation of C-fiber neurons), and other assays known in the art.

Mixtures of agonists and pharmaceutically acceptable salts of any of the foregoing may also be used. See Szallasi and Blumberg, 1999, Pharmacological Reviews 51:159-211, U.S. Pat. No. 5,879,696, and references therein. The concentration of the TRPV1 agonist in the device is between about 0% and about 90% by weight of the drug depot, between about 0% and about 70% by weight of the drug depot, between about 0% and about 50% by weight of the drug depot, between about 0% and about 30% by weight of the drug depot, between about 0% to about 20% of the drug depot, between about 0% and about 10% by weight of the drug depot, between about 0% and about 8% of the drug depot, between about 0% and 6% by weight of the drug depot, between about 0% and 5% by weight of the drug depot, between about 0% and 4% by weight of the drug depot, between about 0% and 2% by weight of the drug depot, or between about 0% and about 1% by weight of the drug depot. In some instances, the concentration of the TRPV1 agonist in the device is 0.04% or less by weight of the drug depot.

It will be appreciated that for a given desired total drug load the percentage of loading may be varied by varying the adhesive matrix thickness and/or concentration of the drug in penetration enhancer or mixture thereof. Also the amount of drug in the adhesive matrix may exceed the desired therapeutic dose to keep the concentration gradient high so that the flux-rate of the drug release from the patch remains constant throughout its intended use. For example, in a device designed to deliver a total of 30 mg of drug over a 24 hour period and then to be replaced by a fresh device, as much as 50 to 100 mg of drug may be included in the device. This ensures high thermodynamic activity of drug at the end of the 24 hour period. For similar reasons excess non-hydrophilic enhancers may also be included in the delivery devices contemplated in this application.

B. Penetration Enhancer/Solvent

Amphiphilic molecules are characterized as having a polar water-soluble group attached to a water-insoluble hydrocarbon chain. In general, amphiphilic penetration enhancers have a polar head group and exhibit appreciable solubility in both aqueous and non-hydrophilic systems. These categories include: surfactants, short chain alcohols, charged quaternary ammonium compounds. Examples of such amphiphilic solvents are butanediols, such as 1,3-butanediol, dipropylene glycol, tetrahydrofurfuryl alcohol, diethylene glycol dimethyl ether, diethylene glycol monoethyl ether, diethylene glycol monobutyl ether, propylene glycol, dipropylene glycol, carboxylic acid esters of tri- and diethylene glycol, polyethoxylated fatty alcohols of 6-18 C atoms or 2,2-dimethyl-4-hydroxymethyl-1,3-dioxolane (Solketal®) or mixtures of these solvents.

Without intending to be bound by any specific theory of operation, penetration enhancers are believed to operate by a variety of mechanisms such as for example increasing the fluidity of membranes, selective perturbation of the intercellular lipid bilayers present in the stratum corneum, opening new polar pathways as indicated by increased electrical conductivity of the tissue. (Eric W. Smith and Howard I. Maibach (1995) In: Percutaneous Penetration Enhancers CRC Press New York, pp. 1-20). Exemplary non-hydrophilic penetration enhancers that may be incorporated in the drug delivery devices described here include, but are not limited to, 1-menthone, isopropyl myristate, caprylic alcohol, lauryl alcohol, oleyl alcohol, isopropyl hexanoate, butyl acetate, methyl valerate, ethyl oleate, d-piperitone, d-pulegone, n-hexane, octanol, myristyl alcohol, methyl nonenoyl alcohol, cetyl alcohol, cetearyl alcohol, stearyl alcohol, myristic acid, stearic acid, and isopropyl palmitate.

Other non-hydrophilic penetration enhancers can be identified using routine assays, e.g., in vitro skin permeation studies on rat, pig or human skin using Franz diffusion cells (see Franz et al., “Transdermal Delivery” In: Treatise on Controlled Drug Delivery. A. Kydonieus. Ed. Marcell Dekker: New York, 1992; pp 341-421). Many other methods for evaluation of enhancers are known in the art, including the high throughput methods of Karande and Mitragotri, 2002, “High throughput screening of transdermal formulations” Pharm Res 19:655-60, and Karande and Mitragotri, 2004, “Discovery of transdermal penetration enhancers by high-throughput screening”).

Non-hydrophilic penetration enhancers suitable for use in the present invention are pharmaceutically acceptable non-hydrophilic penetration enhancers. A pharmaceutically acceptable non-hydrophilic penetration enhancer can be applied to the skin of a human patient without detrimental effects (i.e., has low or acceptable toxicity at the levels used). The non-hydrophilic penetration enhancers employed generally also have ClogP values of 1.0 or higher. Non-hydrophilic penetration enhancers having a ClogP value of greater than or equal to 2.0, greater than or equal to 3.0, greater than or equal to 5.0, greater than or equal to 7.0, or greater than or equal to 9.0 may also be used. Such penetration enhancers include, but are not limited to, enhancers from any of the following classes: fatty long chain alcohols or other alcohols, including phenols and polyols, fatty acids (linear or branched); terpenes (e.g., mono, di and sequiterpenes; hydrocarbons, alcohols, ketones); fatty acid esters, ethers, amides, amines, hydrocarbons.

The hydrophilicity of an amphiphilic penetration enhancer typically makes it incompatible with the adhesive so that incorporation of the enhancer system solely into the adhesive is difficult. The non-hydrophilic enhancer used is generally more hydrophobic in nature and is more compatible with the adhesive. In one variation, applicable to both the reservoir and monolithic type devices, the non-hydrophilic penetration enhancer is located in the drug depot with the therapeutic compound. In another embodiment, the non-hydrophilic penetration enhancer is incorporated into the adhesive layer while the drug is located in the drug depot. Placement of the non-hydrophilic penetration enhancer in the adhesive is often desirable because it puts the enhancer in direct contact with the stratum corneum. In some cases the non-hydrophilic penetration enhancer is loaded into the adhesive as well as into the drug reservoir.

Specific examples of suitable non-hydrophilic solvents and their ClogP values are given in Table 1 below:

TABLE 1
ClogP values of Exemplary Penetration Enhancers

| Non-hydrophilic Penetration | |
|---|---|
| Enhancer | ClogP |
| l-Menthone | 2.83 |
| Isopropyl myristate | 7.59 |
| Caprylic alcohol | 5.13 |
| Lauryl alcohol | 5.06 |
| Oleyl alcohol | 7.74 |
| Isopropyl butyrate | 2.30 |
| Isopropyl hexanoate | 3.36 |
| Butyl acetate | 1.77 |
| Methyl valerate | 1.04 |
| Ethyl oleate | 8.69 |
| d-Piperitone | 2.5 |
| n-Hexane | 3.87 |
| Octanol | 2.94 |
| Mristyl alcohol | 6.03 |
| Cetyl alcohol | 7.17 |
| Cetearyl alcohol | 7.17-8.23 |
| (mixture) | |
| Stearyl alcohol | 8.23 |
| Myristic acid | 6.15 |
| Stearic acid | 8.27 |
| Isopropyl palmitate | 8.65 |

C. The Microreservoir System

As previously mentioned, in one variation, the drug delivery device is a microreservoir system. Polysiloxanes may be used in this type of drug delivery device. Polysiloxanes can be made from solvent-free two-component systems or a solution in organic solvents. For production of drug delivery device, self-adhesive polysiloxanes dissolved in solvents are preferred.

There exist two fundamentally different variants of polysiloxanes: the normal polysiloxane which have free silanol groups as shown in formula 1,

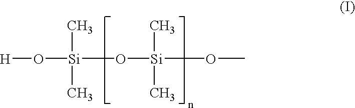

silanol groups are derivatized by trimethylsilyl groups. Such amine-resistant polysiloxanes have also proven suitable for therapeutic compound-containing drug delivery devices without basic therapeutic compounds and/or basic excipients. Formula 1 shows the structure of a linear polysiloxane molecule that is prepared from dimethylsiloxane by polycondensation. Three-dimensional crosslinking can be achieved by the additional use of methylsiloxane.

Other polysiloxanes suitable for use with the methods and devices described herein have the methyl groups completely or partially replaced by other alkyl radicals, or alternatively phenyl radicals.

The solvent or the solvent mixture of the microreservoir system may also contain a viscosity-enhancing additive. Exemplary viscosity-increasing additives include, for example, a cellulose derivative (such as, ethyl cellulose or hydroxypropylcellulose) and a high molecular weight polyacrylic acid or its salt and/or derivatives such as esters.

The proportion of the microreservoir droplets in the matrix is typically less than about 40% by weight, more typically less than about 35% by weight and most typically between about 20 and about 30% by weight.

A mixture of a polysiloxane of medium tack and a polysiloxane of high tack may also be used with the devices and methods described herein. The suitable polysiloxanes for use in the matrix are synthesized from linear bifunctional and branched polyfunctional oligomers, and the ratio of both types of oligomers determines the physical properties of the adhesives. More polyfunctional oligomers result in a more cross-linked adhesive with a higher cohesion and a reduced tack, less polyfunctional oligomers result in a higher tack and a reduced cohesion. For example, the high tack version used in the Examples below is tacky enough to stick on human skin, while the medium tack version is not nearly as tacky, but is useful nevertheless to compensate the softening effect of other ingredients in the device such as, for example, capsaicin and the penetration enhancers in the microreservoirs. A silicone oil (e.g., dimethicone) may be added to increase the adhesive property of the matrix, for example, by using 0.5-5% by weight of the silicone oil.

In one variation, the matrix contains at least about 0.05% to about 10% by weight of capsaicin or capsaicin analog, about 10 to about 25% by weight of oleyl alcohol, about 0% to about 5% by weight of ethyl cellulose, about 0% to about 5% by weight of silicone oil, and about 55% to about 85% by weight of self-adhesive pressure sensitive polysiloxane. The coating weight of the matrix is typically from between about 30 and about 350 g/m², and more typically between about 50 and about 120 g/m². Suitable materials for the backing layer include, for example, a polyester film (e.g., 10-60 μm thick), an ethylene-vinyl acetate copolymer, or the like.

In another variation, a microreservoir type device includes a liquid drug preparation dispersed in an adhesive matrix in the form of small droplets (“microreservoirs”). The appearance of a microreservoir system is similar to a classical matrix system, and a microreservoir system can only be recognized from a typical matrix system with difficulty, since the small microreservoirs can only be recognized under the microscope. In the preceding and the following sections therefore, the therapeutic compound-containing part of the drug delivery device is also described by “matrix”. The size of the resulting droplets depends on the stirring conditions and the applied shear forces during stirring. The size is very consistent and reproducible using the same mixing conditions. The size rage of microreservoir droplets may be from about 1 to about 150 μm, or from about 5 to about 50 μm, or from about 10 to about 30 μm.

It is, however, to be noted that unlike classical matrix systems, in microreservoir systems the therapeutic compound is contained mainly in the microreservoirs (and only to a small extent in the adhesive). In this sense, microreservoir systems can be considered a mixed type of matrix drug delivery device and reservoir drug delivery device and combines the advantages of both drug delivery device variants. As in classical reservoir systems, the saturation solubility can easily be adjusted by the choice of the solvent to a value adequate for the particular requirements, and as in classical matrix systems the drug delivery device can be divided into smaller drug delivery devices using scissors without leakage.

The microreservoir systems described here may also include a diffusion-rate-controlling membrane to control the release of the therapeutic compound and excipient. However, for short application times in which the therapeutic ingredient is rapidly released, a control membrane is usually not present.

One example of a suitable system composition for use with the devices described here is shown in Table 2 below.

TABLE 2
Exemplary composition of a matrix of a microreservoir
system for the topical high-dose delivery of capsaicin

| Component | Percent by weight |
|---|---|
| Capsaicin | 3 |
| Olyel alcohol | 20 |
| Self-adhesive polysiloxane matrix | 77 |

The thickness of the matrix may correspond to a coating weight of about 30 to about 350 g/m², but differing values can also be used depending on the properties of the specific formulation. A matrix thickness of between about 50 and about 100 μm may also be suitable.

Again, the backing layer for the drug delivery device should ideally be relatively impermeable or inert with respect to the drug and the non-hydrophilic solvent selected (e.g., oleyl alcohol). One suitable backing layer is polyester, but other materials such as, for example, ethylene-vinyl acetate copolymers and polyamide are suitable as well. In practice, a polyester film about 51 μm thick has proven highly suitable. In order to improve the adhesion of the matrix to the backing layer, it is advantageous to siliconize the contact side of the backing layer to the matrix. Adhesives based on polyacrylates do not adhere to such siliconized films or adhere relatively poorly, while adhesives based on polysiloxanes, adhere relatively well on account of their chemical similarity to the siliconized films.

The drug delivery devices typically also include a protective film, which protects the device during storage, but is removed before use. Typically, polyester films are used, because once they are surface treated, they are repellent to adhesives based on polysiloxanes. Suitable films are supplied by a number of manufacturers and are known to those having ordinary skill in the art.

II. Methods of Making the Devices

A process for the production of a topical drug delivery device will now be described. Typically, this process comprises dissolving, completely or partially, the therapeutic compound in a non-hydrophilic solvent, adding this solution to a solution of a polysiloxane or the matrix constituents and dispersing with stirring, coating the resulting dispersion onto a protective layer that is removable and removing the solvent of the polysiloxane at elevated temperature, and laminating the backing layer onto the dried layer.

Suitable solvents for adhesives are, for example, petroleum ethers or alkanes such as n-hexane and n-heptane or ethyl acetate. The dispersion of the therapeutic compound solution may be realized more easily if the viscosity of the therapeutic compound solution is increased by the addition of a suitable agent such as, for example, a cellulose derivative such as ethylcellulose or hydroxypropylcellulose. The dispersion is then coated onto the removable protective film in a thickness, which after the removal of the solvent of the adhesive, affords a matrix layer having the desired thickness. The dried layer is then laminated with the backing layer and thus the finished drug delivery device laminate may be obtained.

The drug delivery devices may be punched out of this laminate in the desired shape and size and packed into a suitable sachet of primary packing. A primary packing may be a laminate consisting of paper/glue/aluminum foil/glue/Barex®, as is described in U.S. Pat. No. RE37,934, which is hereby incorporated by reference in its entirety. Barex® is a heat-sealable polymer based on rubber-modified acrylonitrile copolymer, which is distinguished by a low absorptivity for volatile ingredients of drug delivery devices.

Because the microreservoir system typically has no diffusion-rate-controlling membrane controlling the release of therapeutic compound, the only element controlling the release of therapeutic compound into the deeper skin layers may be the skin or the uppermost layer of skin, the stratum corneum. The optimization of the matrix composition can be therefore carried out by in vitro permeation studies using human skin and by Franz diffusion cells as described in Venter et al., 2001, “A comparative study of an in situ adapted diffusion cell and an in vitro Franz diffusion cell method for transdermal absorption of doxylamine” Eur J Pharm Sci, 13:169-77.

### EXAMPLES

The following examples serve to more fully describe the manner of making and using the above-described drug delivery devices. It is understood that these examples are provided for illustrative purposes only and should not be construed as limiting the scope of the invention.

### Example 1

### Preparation of a Microreservoir Device Containing 0.04% Capsaicin by Weight in the Drug Depot

To 80 mg of capsaicin, 16.0 grams of oleyl alcohol was added and the components were mixed. Ethyl cellulose, 200 mg, was then added and mixed thoroughly and set aside for two hours. Bio-PSA® 4201, 36.74 grams and Bio-PSA® 4301, 146.98 grams, were added and the adhesive mass was mixed vigorously until gelled mixture of olyel alcohol, capsaicin, and ethyl cellulose was uniformly dispersed as fine globules in the adhesive. The resulting adhesive matrix was subsequently coated on a release liner 3M™ Scotchpak™ 1022, and solvent n-heptane was dried by blowing hot air at a temperature between 35 to 40° C. Coating weight after the removal of the n-heptane was approximately 273.6 g/m². The dried film was then laminated with the polyester backing layer, 3M™ Scotchpak™ 9733, and the finished drug delivery device was punched out (5 cm×5 cm). The punched drug delivery devices were then sealed into a sachet of a primary packing laminate.

### Example 2

### Preparation of a Microreservoir Device Containing 2.0% Capsaicin by Weight in the Drug Depot

To 4.0 grams of capsaicin, 20.0 grams of olyel alcohol was added and the components were mixed. Ethyl cellulose, 200 mg, was then added and mixed thoroughly and set aside for two hours. Bio-PSA® 4301, 175.80 grams was added and the adhesive mass was mixed vigorously until gelled mixture of olyel alcohol, capsaicin, and ethyl cellulose was uniformly dispersed as fine globules in the adhesive. The resulting adhesive matrix was subsequently coated on a release liner 3M™ Scotchpak™ 1022, and solvent n-heptane was dried by blowing hot air at a temperature between 35 to 40° C. Coating weight after the removal of the n-heptane was approximately 277.9 g/m². The dried film was then laminated with the polyester backing layer, 3M™ Scotchpak™ 9733, and the finished drug delivery device was punched out (5 cm×5 cm). The punched drug delivery devices were then sealed into a sachet of a primary packing laminate.

### Example 3

### Preparation of a Microreservoir Device Containing 4% Capsaicin by Weight in the Drug Depot

To 8.0 grams of capsaicin, 36.0 grams of olyel alcohol was added and the components were mixed. Ethyl cellulose, 2.0 grams, was then added and mixed thoroughly and set aside for two hours. Bio-PSA® 4301, 154.0 grams was added and the adhesive mass was mixed vigorously until gelled mixture of olyel alcohol, capsaicin, and ethyl cellulose was uniformly dispersed as fine globules in the adhesive. The resulting adhesive matrix was subsequently coated on a release liner 3M™ Scotchpak™ 1022, and solvent n-heptane was dried by blowing hot air at a temperature between 35 to 40° C. Coating weight after the removal of the n-heptane was approximately 218.4 g/m². The dried film was then laminated with the polyester backing layer, 3M™ Scotchpak™ 9733, and the finished drug delivery device was punched out (5 cm×5 cm). The punched drug delivery devices were then sealed into a sachet of a primary packing laminate.

### Example 4

### Preparation of a Microreservoir Device Containing 6% Capsaicin by Weight in the Drug Depot

To 12.0 grams of capsaicin, 40.0 grams of olyel alcohol was added and the components were mixed. Ethyl cellulose, 4.0 grams, was then added and mixed thoroughly and set aside for two hours. Bio-PSA® 4301, 144.0 grams was added and the adhesive mass was mixed vigorously until gelled mixture of olyel alcohol, capsaicin, and ethyl cellulose was uniformly dispersed as fine globules in the adhesive. The resulting adhesive matrix was subsequently coated on a release liner 3M™ Scotchpak™ 1022, and solvent n-heptane was dried by blowing hot air at a temperature between 35 to 40° C. Coating weight after the removal of the n-heptane was approximately 245.0 g/m². The dried film was then laminated with the polyester backing layer, 3M™ Scotchpak™ 9733, and the finished drug delivery device was punched out (5 cm×5 cm). The punched drug delivery devices were then sealed into a sachet of a primary packing laminate.

### Example 5

### Preparation of a Microreservoir Device Containing 8% Capsaicin by Weight in the Drug Depot

To 16.0 grams of capsaicin, 44.0 grams of olyel alcohol was added and the components were mixed. Ethyl cellulose, 4.0 grams, was then added and mixed thoroughly and set aside for two hours. Bio-PSA® 4301, 136.0 grams was added and the adhesive mass was mixed vigorously until gelled mixture of olyel alcohol, capsaicin, and ethyl cellulose was uniformly dispersed as fine globules in the adhesive. The resulting adhesive matrix was subsequently coated on a release liner 3M™ Scotchpak™ 1022, and solvent n-heptane was dried by blowing hot air at a temperature between 35 to 40° C. Coating weight after the removal of the n-heptane was approximately 352.9 g/m². The dried film was then laminated with the polyester backing layer, 3M™ Scotchpak™ 9733, and the finished drug delivery device was punched out (5 cm×5 cm). The punched drug delivery devices were then sealed into a sachet of a primary packing laminate.

### Example 6

### Preparation of a Microreservior Device Containing 10% Capsaicin by Weight in the Drug Depot

To 20.0 grams of capsaicin, 50.0 grams of olyel alcohol was added and the components were mixed. Ethyl cellulose, 4.0 grams, was then added and mixed thoroughly and set aside for two hours. Bio-PSA® 4301, 126.0 grams was added and the adhesive mass was mixed vigorously until gelled mixture of olyel alcohol, capsaicin, and ethyl cellulose was uniformly dispersed as fine globules in the adhesive. The resulting adhesive matrix was subsequently coated on a release liner 3M™ Scotchpak™ 1022, and solvent n-heptane was dried by blowing hot air at a temperature between 35 to 40° C. Coating weight after the removal of the n-heptane was approximately 81.8 g/m². The dried film was then laminated with the polyester backing layer, 3M™ Scotchpak™ 9733, and the finished drug delivery device was punched out (5 cm×5 cm). The punched drug delivery devices were then sealed into a sachet of a primary packing laminate.

### Example 7

### Preparation of a Monolithic Device Containing 0.04% Capsaicin by Weight in the Drug Depot

To 1.2 mg of capsaicin, 1000 mg of olyel alcohol was added and the components were mixed. Gelatin, 1999 mg, was then added and mixed thoroughly. Polyester backing layer, 3M™ Scotchpak™ 9733 was heat-sealed with 3M™ CoTran™ 9712 to make 5 cm×5 cm pouch with one end open. The polyester backing layer extended beyond boundaries of the pouch by about 1 cm on all sides. The above mixed contents were filled into the pouch, and rolled to make a layer of uniform thickness that extended to the edges of the pouch. The open side then heat-sealed. The polyester backing layer extended outside the pouch was then coated with a thin layer of Bio-PSA® 4201 and subsequently dried by blowing hot air at a temperature between 35 to 40° C. The dried adhesive film was then laminated with a 6 cm×6 cm piece of release liner Scotchpak™ 1022. The finished drug delivery device was then sealed into a sachet of a primary packing laminate.

### Example 8

### Preparation of a Monolithic Device Containing 2% Capsaicin by Weight in the Drug Depot

To 60 mg of capsaicin, 1000 mg of olyel alcohol was added and the components were mixed. Gelatin, 1940 mg, was then added and mixed thoroughly. Polyester backing layer, 3M™ Scotchpak™ 9733 was heat-sealed with 3M™ CoTran™ 9712 to make 5 cm×5 cm pouch with one end open. The polyester backing layer extended beyond boundaries of the pouch by about 1 cm on all sides. The above mixed contents were filled into the pouch, and rolled to make a layer of uniform thickness that extended to the edges of the pouch. The open side then heat-sealed. The polyester backing layer extended outside the pouch was then coated with a thin layer of Bio-PSA® 4201 and subsequently dried by blowing hot air at a temperature between 35 to 40° C. The dried adhesive film was then laminated with a 6 cm×6 cm piece of release liner Scotchpak™ 1022. The finished drug delivery device was then sealed into a sachet of a primary packing laminate.

### Example 9

### Preparation of a Monolithic Device Containing 4% Capsaicin by Weight in the Drug Depot

To 120 mg of capsaicin, 1000 mg of olyel alcohol was added and the components were mixed. Gelatin, 1880 mg, was then added and mixed thoroughly. Polyester backing layer, 3M™ Scotchpak™ 9733 was heat-sealed with 3M™ CoTran™ 9712 to make 5 cm×5 cm pouch with one end open. The polyester backing layer extended beyond boundaries of the pouch by about 1 cm on all sides. The above mixed contents were filled into the pouch, and rolled to make a layer of uniform thickness that extended to the edges of the pouch. The open side then heat-sealed. The polyester backing layer extended outside the pouch was then coated with a thin layer of Bio-PSA® 4201 and subsequently dried by blowing hot air at a temperature between 35 to 40° C. The dried adhesive film was then laminated with a 6 cm×6 cm piece of release liner Scotchpak™ 1022. The finished drug delivery device was then sealed into a sachet of a primary packing laminate.

### Example 10

### Preparation of a Monolithic Device Containing 6% Capsaicin by Weight in the Drug Depot

To 180 mg of capsaicin, 1000 mg of olyel alcohol was added and the components were mixed. Gelatin, 1820 mg, was then added and mixed thoroughly. Polyester backing layer, 3M™ Scotchpak™ 9733 was heat-sealed with 3M™ CoTran™ 9712 to make 5 cm×5 cm pouch with one end open. The polyester backing layer extended beyond boundaries of the pouch by about 1 cm on all sides. The above mixed contents were filled into the pouch, and rolled to make a layer of uniform thickness that extended to the edges of the pouch. The open side then heat-sealed. The polyester backing layer extended outside the pouch was then coated with a thin layer of Bio-PSA® 4201 and subsequently dried by blowing hot air at a temperature between 35 to 40° C. The dried adhesive film was then laminated with a 6 cm×6 cm piece of release liner Scotchpak™ 1022. The finished drug delivery device was then sealed into a sachet of a primary packing laminate.

### Example 11

### Preparation of a Monolithic Device Containing 8% Capsaicin by Weight in the Drug Depot

To 240 mg of capsaicin, 1000 mg of olyel alcohol was added and the components were mixed. Gelatin, 1760 mg, was then added and mixed thoroughly. Polyester backing layer, 3M™ Scotchpak™ 9733 was heat-sealed with 3M™ CoTran™ 9712 to make 5 cm×5 cm pouch with one end open. The polyester backing layer extended beyond boundaries of the pouch by about 1 cm on all sides. The above mixed contents were filled into the pouch, and rolled to make a layer of uniform thickness that extended to the edges of the pouch. The open side then heat-sealed. The polyester backing layer extended outside the pouch was then coated with a thin layer of Bio-PSA® 4201 and subsequently dried by blowing hot air at a temperature between 35 to 40° C. The dried adhesive film was then laminated with a 6 cm×6 cm piece of release liner Scotchpak™ 1022. The finished drug delivery device was then sealed into a sachet of a primary packing laminate.

### Example 12

### Preparation of a Monolithic Device Containing 10% Capsaicin by Weight in the Drug Depot

To 300 mg of capsaicin, 1000 mg of olyel alcohol was added and the components were mixed. Gelatin, 1700 mg, was then added and mixed thoroughly. Polyester backing layer, 3M™ Scotchpak™ 9733 was heat-sealed with 3M™ CoTran™ 9712 to make 5 cm×5 cm pouch with one end open. The polyester backing layer extended beyond boundaries of the pouch by about 1 cm on all sides. The above mixed contents were filled into the pouch, and rolled to make a layer of uniform thickness that extended to the edges of the pouch. The open side then heat-sealed. The polyester backing layer extended outside the pouch was then coated with a thin layer of Bio-PSA® 4201 and subsequently dried by blowing hot air at a temperature between 35 to 40° C. The dried adhesive film was then laminated with a 6 cm×6 cm piece of release liner Scotchpak™ 1022. The finished drug delivery device was then sealed into a sachet of a primary packing laminate.

### Example 13

### Preparation of a Monolithic Device Containing 4% Capsaicin by Weight in the Drug Depot

To 120 mg of capsaicin, 1000 mg of olyel alcohol was added and the components were mixed. Ethyl cellulose, 1880 mg, was then added and mixed thoroughly. Polyester backing layer, 3M™ Scotchpak™ 9733 was heat-sealed with 3M™ CoTran™ 9712 to make 5 cm×5 cm pouch with one end open. The polyester backing layer extended beyond boundaries of the pouch by about 1 cm on all sides. The above mixed contents were filled into the pouch, and rolled to make a layer of uniform thickness that extended to the edges of the pouch. The open side then heat-sealed. The polyester backing layer extended outside the pouch was then coated with a thin layer of Bio-PSA® 4201 and subsequently dried by blowing hot air at a temperature between 35 to 40° C. The dried adhesive film was then laminated with a 6 cm×6 cm piece of release liner Scotchpak™ 1022. The finished drug delivery device was then sealed into a sachet of a primary packing laminate.

### Example 14

### In Vitro Dissolution Assays

Microreservoir Type of Delivery Device. The release liners were removed from the patches described in examples 1-6 and mounted onto a glass plate (6 cm×6 cm) with a doubled side adhesive tape such that one side on the tape was adhered to the glass plate and other side to the backing layer of the patch. The six glass plates were immersed in 200 mL DI water containing 0.1% w/v sodium azide such that patches were exposed to the aqueous medium without touching the container. The container were tightly capped and mounted onto a shaker. The shaking was gentle horizontal oscillations and did not involve tumbling. The solutions were sampled (200 μL sample size) at 30 min, 1 hour, 3 hours and 18 hours and analyzed on HPLC for capsaicin content. The capsaicin release results are listed below in Table 3.

TABLE 3
Capsaicin Release from Microreservoir Type Patches

| Capsaicin Concentration | | | | |
|---|---|---|---|---|
| (w/w %) In Drug Depot | Amount of Capsaicin Released (μg) | | | |
| of Patch | 30 Min. | 1 Hr. | 3 Hrs. | 18 Hrs. |
| 0.04% | 27.41 | 37.61 | 60.56 | 115.49 |
| 2.0% | 425.24 | 630.75 | 1155.67 | 2566.86 |
| 4.0% | 1018.28 | 1520.25 | 2608.13 | 4241.12 |
| 6.0% | 1855.01 | 2968.48 | 5188.55 | 6725.11 |
| 8.0% | 2530.57 | 4162.67 | 7352.07 | 7845.59 |
| 10.0% | 1844.41 | 2986.90 | 5461.02 | 6871.52 |

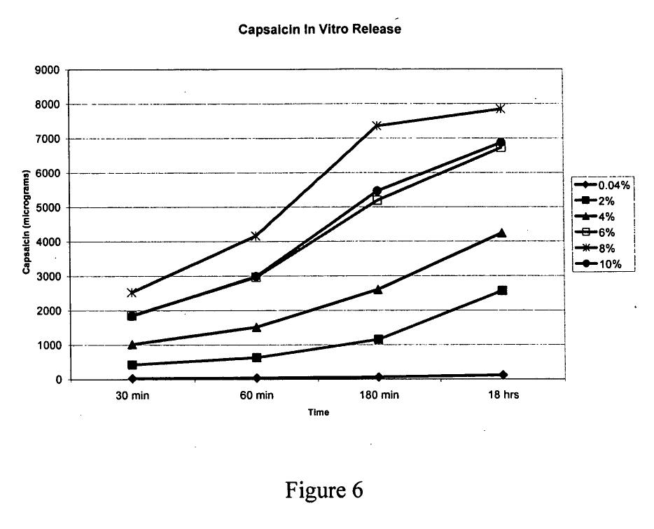

FIG. 6 shows that amount of capsaicin released is linear with time as well as with concentration of the capsaicin in the patch. It should be noted that low amount of capsaicin released from 10% w/w patch relative to 8% w/w patch is due to relatively thin coating on 10% w/w patch (compare examples 5 and 6 above).

Monolithic Type of Delivery Device. The release liners were removed from the patches described in examples 7-12 and mounted onto a glass plate (6 cm×6 cm) with a doubled side adhesive tape such that one side on the tape was adhered to the glass plate and other side to the backing layer of the patch. The six glass plates were immersed in 200 mL DI water containing 0.1% w/v sodium azide such that patches were exposed to the aqueous medium without touching the container. The container were tightly capped and mounted onto a shaker. The shaking was gentle horizontal oscillations and did not involve tumbling. The solutions were sampled (200 μL sample size) at 30 min, 1 hour, 3 hours and 24 hours and analyzed on HPLC for capsaicin content. The capsaicin release results are listed below in Table 4.

TABLE 4
Capsaicin Release from Monolithic Type Patches

| Capsaicin Concentration | | | | |
|---|---|---|---|---|
| (w/w %) In Drug Depot | Amount of Capsaicin Released (μg) | | | |
| of Patch | 30 Min. | 1 Hr. | 3 Hrs. | 24 Hrs. |
| 0.04% | 2.70 | 4.39 | 11.29 | 88.93 |
| 2.0% | 57.59 | 86.39 | 206.53 | 1400.50 |
| 4.0% | 56.43 | 93.01 | 255.73 | 1771.37 |
| 6.0% | 70.97 | 117.70 | 323.31 | 5059.27 |
| 8.0% | 69.70 | 124.65 | 375.89 | 1699.12 |
| 10.0% | 115.42 | 167.40 | 414.98 | 3983.10 |

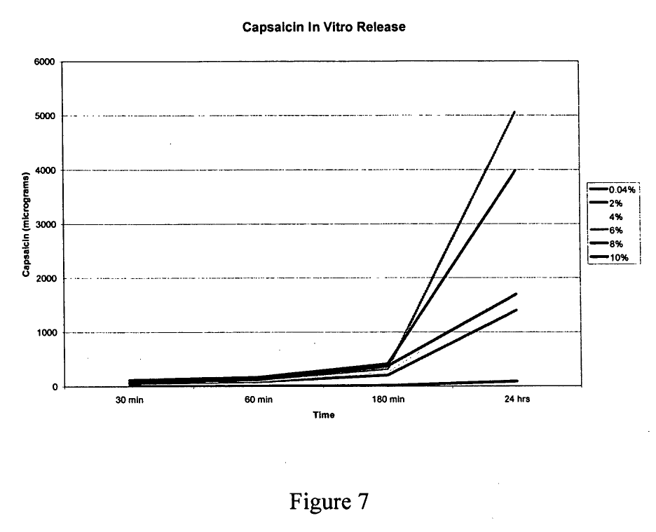

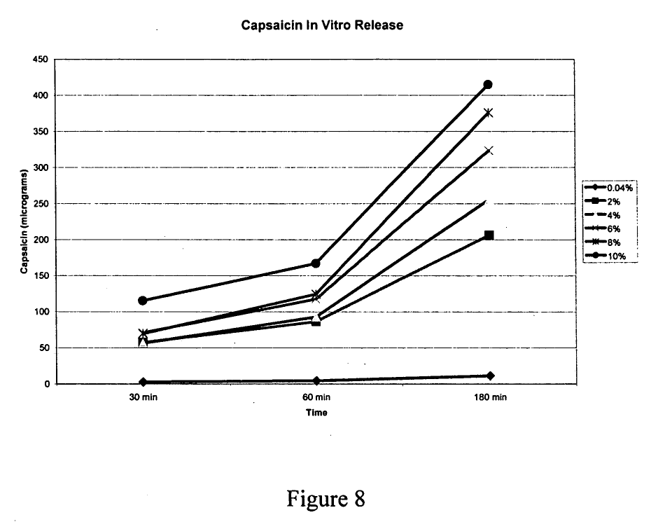

Again in case of monolithic type of patches, FIG. 7 shows that amount of capsaicin released is linear with time as well as with concentration of the capsaicin in the patch. It should be noted that relative to microreservoir type of patches, a low amount of capsaicin released from monolithic type of patches is due as expected due to the presence of a diffusion-rate-controlling membrane.

What is claimed as new and desired to be protected by Letters Patent of the United States is:

1. A drug delivery device comprising:
a) a drug depot having a therapeutically effective amount of a TRPV1 agonist;
b) a non-hydrophilic penetration enhancer having a ClogP value greater than 1.0; and
c) an occlusive backing.

2. The drug delivery device of claim 1 wherein the active agent is selected from the group consisting of capsaicin, capsaicinoids, capsaicin analogs, capsaicin derivatives, and combinations thereof.

3. The drug delivery device of claim 2 wherein the TRPV1 agonist comprises capsaicin.

4. The drug delivery device of claim 2 wherein the TRPV1 agonist comprises a capsaicinoid.

5. The drug delivery device of claim 2 wherein the TRPV1 agonist comprises a capsaicin analog.

6. The drug delivery device of claim 2 wherein the TRPV1 agonist comprises a capsaicin derivative.

7. The drug delivery device of claim 1 wherein the TRPV1 agonist comprises at least about 30% of the drug depot by weight.

8. The drug delivery device of claim 1 wherein the TRPV1 agonist comprises at least about 20% of the drug depot by weight.

9. The drug delivery device of claim 1 wherein the TRPV1 agonist comprises at least about 10% of the drug depot by weight.

10. The drug delivery device of claim 1 wherein the TRPV1 agonist comprises at least about 8% of the drug depot by weight.

11. The drug delivery device of claim 1 wherein the TRPV1 agonist comprises at least about 6% of the drug depot by weight.

12. The drug delivery device of claim 1 wherein the TRPV1 agonist comprises at least about 5% of the drug depot by weight.

13. The drug delivery device of claim 1 wherein the TRPV1 agonist comprises at least about 4% of the drug depot by weight.

14. The drug delivery device of claim 1 wherein the TRPV1 agonist comprises at least about 2% of the drug depot by weight.

15. The drug delivery device of claim 1 wherein the TRPV1 agonist comprises at least about 0.04% of the drug depot by weight.

16. The drug delivery device of claim 1 wherein the non-hydrophilic penetration enhancer is selected from the group consisting of 1-menthone, isopropyl myristate, dimethyl isosorbide, caprylic alcohol, lauryl alcohol, oleyl alcohol, isopropyl butyrate, isopropyl hexanoate, butyl acetate, methyl acetate, methyl valerate, ethyl oleate, d-piperitone, d-pulogene, n-hexane, citric acid, ethanol, propanol, isopropanol, ethyl acetate, methyl propionate, methanol, butanol, tert-butanol, octanol, myristyl alcohol, methyl nonenoyl alcohol, cetyl alcohol, cetearyl alcohol, stearyl alcohol, myristic acid, stearic acid, isopropyl palmitate, and combinations thereof.

17. The drug delivery device of claim 16 wherein the non-hydrophilic penetration enhancer comprises oleyl alcohol.

18. The drug delivery device of claim 16 wherein the non-hydrophilic penetration enhancer comprises 1-menthone.

19. The drug delivery device of claim 1 wherein the non-hydrophilic penetration enhancer has a ClogP value greater than or equal to 2.0.

20. The drug delivery device of claim 1 wherein the non-hydrophilic penetration enhancer has a ClogP value greater than or equal to 3.0.

21. The drug delivery device of claim 1 wherein the non-hydrophilic penetration enhancer has a ClogP value greater than or equal to 5.0.

22. The drug delivery device of claim 1 wherein the non-hydrophilic penetration enhancer has a ClogP value greater than or equal to 7.0.

23. The drug delivery device of claim 1 wherein the non-hydrophilic penetration enhancer has a ClogP value greater than or equal to 9.0.

24. The drug delivery device of claim 1 wherein the non-hydrophilic penetration enhancer comprises at least about 35% of the drug depot by weight.

25. The drug delivery device of claim 1 wherein the non-hydrophilic penetration enhancer comprises at least about 30% of the drug depot by weight.

26. The drug delivery device of claim 1 wherein the non-hydrophilic penetration enhancer comprises at least about 25% of the drug depot by weight.

27. The drug delivery device of claim 1 wherein the non-hydrophilic penetration enhancer comprises at least about 20% of the drug depot by weight.

28. The drug delivery device of claim 1 wherein the non-hydrophilic penetration enhancer comprises at least about 15% of the drug depot by weight.

29. The drug delivery device of claim 1 wherein the non-hydrophilic penetration enhancer comprises at least about 10% of the drug depot by weight.

30. The drug delivery device of claim 1 wherein the non-hydrophilic penetration enhancer comprises at least about 5% of the drug depot by weight.

31. The drug delivery device of claim 1 wherein the TRPV1 agonist is dissolved, partially dissolved, or dispersed within the drug depot.

32. The drug delivery device of claim 1 wherein the drug depot comprises a polymeric matrix.

33. The drug delivery device of claim 32 wherein the polymeric matrix comprises an adhesive matrix.

34. The drug delivery device of claim 32 wherein the polymeric matrix comprises a polymer selected from the group consisting of gelatin, polyacrylates, polyisobutylenes, polysiloxanes, polyurethanes, polyvinylpyrrolidones, and co-polymers and combinations thereof.

35. The drug delivery device of claim 1 wherein the drug depot comprises the TRPV1 agonist dissolved or partially dissolved within microreservoirs.

36. The drug delivery device of claim 1 wherein the drug depot comprises the TRPV1 agonist in a liquid reservoir.

37. The drug delivery device of claim 1 further comprising a diffusion-rate-controlling membrane.

38. A method for treating pain or skin condition comprising:
a) applying a drug delivery device to the skin or mucous membrane of a subject, wherein the drug delivery device comprises:
a) a TRPV1 agonist;
b) a non-hydrophilic penetration enhancer having a ClogP value greater than 1; and
c) an occlusive backing, and
b) delivering a therapeutically effective amount of the TRPV1 agonist to alleviate the pain or skin condition.

39. The method of claim 38 wherein the TRPV1 agonist comprises capsaicin.
